(19) United States
(12) Patent Application Publication
(10) Pub. No.: US 2008/0039888 A1
(43) Pub. Date: Feb. 14, 2008

(54) System for Occluding a Blood Vessel, Especially After Artery Catheterization

(76) Inventors: Michel Doare, Frankfurt a.M. (DE); Gerd Stumpp, Hirrlingen (DE); Albertus Scheule, Tubingen (DE)

Correspondence Address: CHRISTIE, PARKER & HALE, LLP, PO BOX 7068, PASADENA, CA, 91109-7068, US

(21) Appl. No.: 11/587,719

(22) PCT Filed: Apr. 21, 2005
(86) PCT No.: PCT/EP05/04287

§ 371 Date: Jul. 18, 2007

(30) Foreign Application Priority Data
May 3, 2004 (DE) 10 2004 022 780.2

(51) Int. Cl. A61M 29/02

(52) U.S. Cl. 606/194

(57) ABSTRACT

A system for occluding a blood vessel at a site of puncture of a blood vessel, especially after artery catheterization, comprises an oblong occlusive means (13) that has a shank (14) and a compression surface (15) configured thereon as the end face. The shank comprises a continuous bore (17) that extends through the compression surface to accommodate at least one guide means (11) inserted into the blood vessel and is, on the distal end (20) opposite the compression surface, adapted to be fixed on the patient on the skin when the compression surface is advanced towards the site of puncture (2).

### CROSS-REFERENCE TO RELATED APPLICATION(S)

This application is a National Phase patent application of International Application Number PCT/EP2005/004287, filed on Apr. 21, 2005, which claims priority of DE 10 2004 022 780.2 filed May 3, 2004.

The invention relates to a system for occluding a blood vessel at a site of puncture of a blood vessel, especially after artery catheterization.

The endovascular diagnosis and therapy of cardiac and vascular diseases almost always necessitates access to the arterial system via the inguinal arteries. After puncture of the artery, a vascular sheath is introduced, via which the diagnosis or therapy catheters are placed. After the end of the intervention, this sheath is removed and pressure is exerted percutaneously on the site of puncture from the outside in order to seal it by compression with the help of natural coagulation.

This customary procedure includes, initially, relatively long manual compression of the tissue containing the site of puncture, followed by several hours of compression by means of a bandage. Not only, but primarily, in adipose patients, with this method, adequate pressure cannot be focused on the site of puncture, such that, occasionally, there is increased bleeding into the perivascular tissue. These hematomas are threatening to the patient from several standpoints:

Loss of blood may result in circulatory instability,

The patient requires blood transfusions with the known risk of infection,

The hematoma is painful, increases the risk of local infection, and is only very slowly resorbed by the body,

In the region of the hematoma, resorption causes tissue inflammation with changes that render further local interventions or operations much more difficult, and

Bleeding is not stopped by the hematoma that develops and the site of puncture must be sutured in an emergency procedure.

All the above mentioned scenarios necessitate a relatively long monitoring outlay and are disadvantageous both to the patient and economically. A relatively long hospitalization of the patient is not uncommon.

From WO 02/072188, a system is known for hemostasis of an artery that has a puncture after arterial catheterization. For this, an apparatus is used that has an elongated flexible hollow shaft that can be inserted into the artery through a catheter introducer. On its forward end, the shank carries an anchor balloon and, axially offset therefrom, a vascular sealing balloon. It is operated in that the shaft is pushed via the catheter introducer far enough into the artery that the anchor balloon is pushed out of the catheter introducer to lie in the artery. After inflation of the anchor balloon, the shaft and the catheter introducer are retracted until the inflated anchor balloon lies against the inside wall of the artery, whereupon the catheter introducer is withdrawn.

Next, the extravascular balloon is inflated while the anchor balloon is deflated and the shank retracted far enough that its tip no longer is inside the artery while the site of puncture remains sealed by the vessel sealing balloon thus inflated. After the puncture at the site of puncture is sealed by natural coagulation, the vessel sealing balloon is likewise deflated and the entire apparatus is withdrawn from the intervention channel. This apparatus is expensive and its operation requires a considerable degree of delicate caution.

The object of the invention is, consequently, to provide a system which reliably enables prevention of protracted bleeding and large hematomas after the puncture of a vessel, in particular an artery, in a relatively simple manner.

To accomplish this object, the system according to the invention has the characteristics of claim 1.

The new system operates with an oblong occlusive means that has a shank and a compression surface configured thereon as the end face, whereby the shank comprises a continuous bore that extends through the compression surface to accommodate at least one guide means inserted into the blood vessel, and is, on the distal end opposite the compression surface, adapted to be fixed on the patient on the skin when the compression surface is advanced towards the site of puncture.

The oblong occlusive means makes it possible to optimize the extravascular pressure on the site of puncture after the puncture of a vessel, in particular an arterial vessel since the compression surface can be placed, using the guide means, for example, a Seldinger wire, in position relative to the site of puncture in the immediate vicinity of the outer vessel wall, with the shank protruding into the skin through the incision point making it possible to exert locally concentrated, precisely measured pressure on the tissue in the region of the site of puncture such that reliable occlusion of the puncture in the vessel wall is obtained. The temporal duration of the pressure action may be selected at will since the shank of the occlusive means can be fixed in a simple manner on the skin level, for instance, by a dressing or a bandage. After coagulation of the blood at the site of puncture, the occlusive means can be retracted and removed simply. The occlusion means itself is economical to produce such that from this standpoint as well there is a reduction in the treatment costs associated with a catheterization.

The shank of the occlusive means is preferably designed substantially in the shape of a cylinder; however, other purpose-appropriate cross-sectional shapes, e.g., oval, may be used. It is also conceivable to design the shank with longitudinal ribs or depressions or with a profile that yields special flexibility characteristics. It is also expedient for the shaft to have an area with an enlarged cross-sectional surface that carries the compression surface. Furthermore, the shank may, for example, be designed like a piston and have a substantially smooth-walled shank portion that connects to the area of enlarged diameter. Starting from the compression surface formed on the end face, the area of enlarged diameter can transition continuously over the length of the shank into the smooth-walled shank portion. The size of the compression surface is thus independent of the cross-sectional area of the shank such that the two elements can be selected purpose-appropriately. In principle, embodiments are also possible in which the compression surface is formed on a shank that has a substantially equal diameter over its entire length.

The compression surface may be substantially circular in shape; however, it is often advantageous for the compression surface to be oblong in order to better adapt the pressure area to the course of the vessel and to the possibly elongated site of puncture. Usually, the compression surface is designed running substantially perpendicular to the longitudinal axis of the shank; however, embodiments are also conceivable in which the compression surface runs at an angle deviating from 90° oblique to the longitudinal axis of the shank, thus taking into account the fact that the access channel to the vessel usually forms an acute angle with the longitudinal axis of the vessel.

The compression site is usually flat but may also be, depending primarily on the region, rounded (convex) or concave or textured.

To enable adaptation to the anatomical characteristics of different patients in the vicinity of the site of puncture, the shank can be designed variable in its length. For this, the shank may, for instance, be designed telescopically or have two parts connected to each other by screw threads. It is also conceivable for the shaft to be designed with predetermined breaking points positioned at intervals relative to each other in the longitudinal direction of the shank that enable bringing the shank to a purpose-appropriate length by breaking off a section of the shank protruding beyond the skin of the patient when the occlusion means is used. Obviously, other designs that enable a purpose-appropriate adjustment of shank length in a simple manner are also conceivable.

In order to seal the bore passing through the shank to accommodate the guide means during placement of the occlusion means after removal of the guide means, a sealing means may be provided for the bore of the shank, which, is designed, for instance, in the form of a plug. The sealing means may have a portion introducible into the bore of the shank that substantially fills the bore over at least a section of its longitudinal extension. Thus, if necessary, it is possible to prevent a column of coagulated blood from remaining in the bore. The sealing means may, however, also be in the form of a cap on the shank. In practice, the bore may optionally also be simply sealed by a cotton styptic or the like.

It may also be expedient for the shank to have, on its distal end, a widening that

is, for instance, designed with a cap-like shape and is attached to the shank as one piece or is removable. In the latter case, the cap-like widening may simultaneously form the sealing means for the bore in the shank. The widening on the distal shank end yields, in any case, a large support area for a dressing fixing the occlusive means in the skin area, perhaps in the form of a bandage, a dressing, or the like.

Finally, embodiments are also conceivable in which the occlusive means has a balloon in the vicinity of the compression surface, which is inflatable via a line running through the shank, and it permits the compression surface to expand after placement of the occlusive means and to generate an additional pressure effect. The balloon may be arranged, in the uninflated state, at least partially, in a recess in the region of the compression surface and/or in the bore of the shank in order to thus facilitate the introduction of the compression means into the introduction channel present in the tissue.

Additional advantageous characteristics and embodiments of the system according to the invention for intracorporeal maximization of pressure for the occlusion of blood vessels, in particular after artery catheterization, are the subject matter of dependent claims and are obvious from the following description of exemplary embodiments of the object of the invention.

The drawings depict exemplary embodiments of the object of the invention. They depict:

FIG. 1 a partial schematic depiction of the arterial blood vessel system of a patient showing the femoral artery access region for cardiac catheterization,

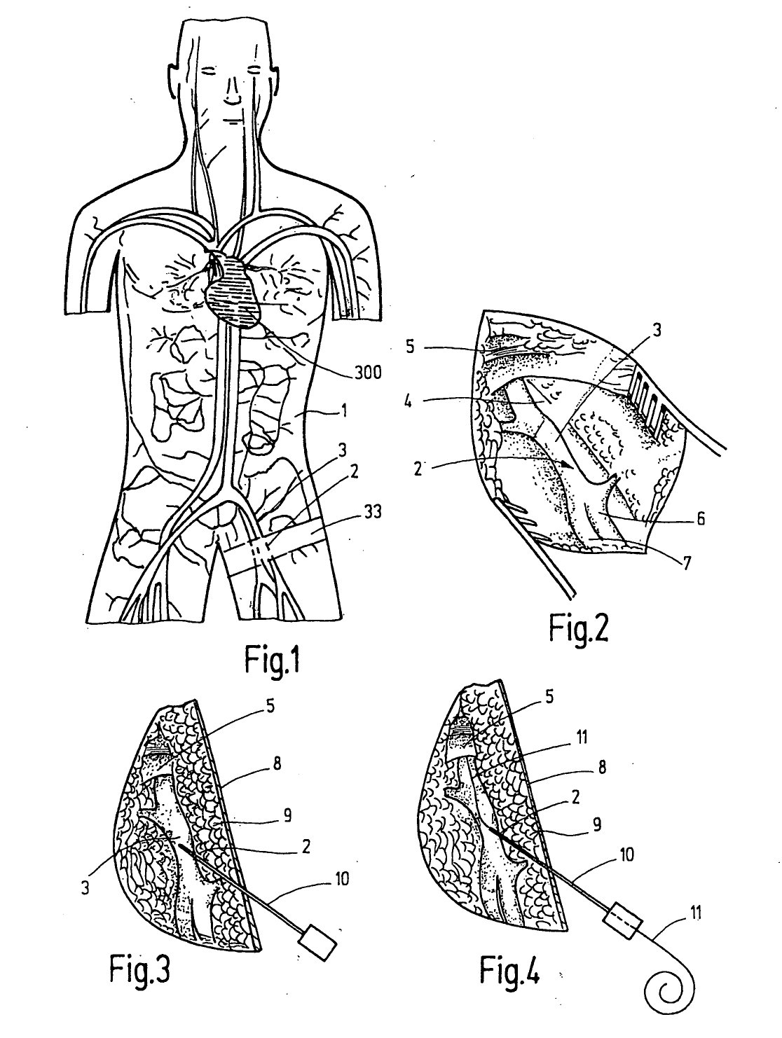

FIG. 2 an exposed femoral artery access region of the depiction of FIG. 1, showing a puncture site on the ventral side of the Arteria femoralis communis,

FIG. 3 through 9

i) in each case, a longitudinal depiction of the thigh at the puncture site depicted in FIG. 2, showing significant steps in the puncturing of the Arteria femoralis communis in connection with a catheterization and the subsequent occlusion of the puncture site with the vascular occlusion system according to the invention,

FIG. 10 an occlusive means of the vascular occlusion system according to the invention in a first embodiment in a schematic depiction, greatly enlarged,

FIG. 11 the occlusive means of FIG. 1 in a top plan view of the compression surface,

FIG. 12 the occlusive means of a vascular occlusion system according to the invention in a second embodiment, in a different scale, and a partial cross-sectional depiction corresponding to FIG. 10,

FIG. 13 the occlusive means of FIG. 12 in a top plan view of the compression surface,

FIG. 14 the occlusive means of a vascular occlusion system according to the invention in a third embodiment, in a schematic longitudinal section similar to FIG. 12 and in a corresponding scale,

FIG. 15 the occlusive means of FIG. 13 in a top plan view of the compression surface,

FIG. 16 the occlusive means of a vascular occlusion system according to the invention in a fourth embodiment with a shank variable in length, in a schematic longitudinal section similar to FIG. 12 and in a corresponding scale,

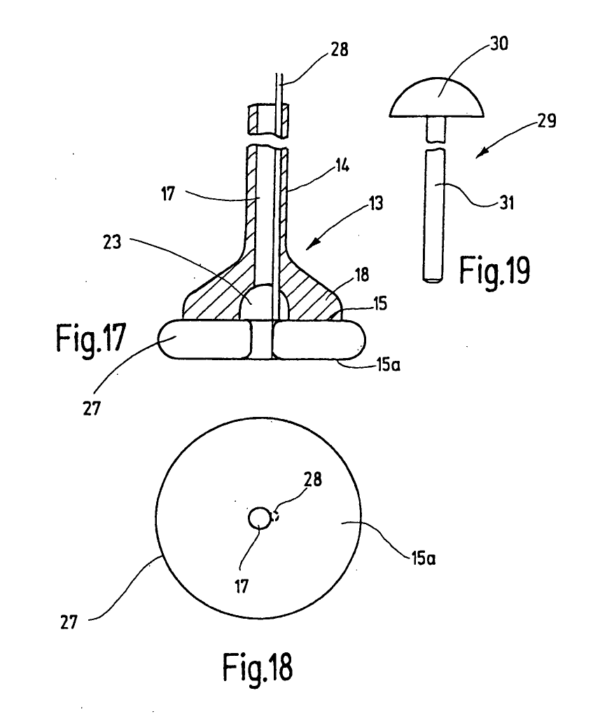

FIG. 17 the occlusive means of a vascular occlusion system according to the invention in a fifth embodiment, in a schematic longitudinal section similar to FIG. 12, showing a balloon arranged in the region of the compression surface in the inflated state,

FIG. 18 the occlusive means of FIG. 17 in the top plan view of the inflated balloon, and

FIG. 19 a sealing means for one of the occlusion means of one of FIGS. 10 through 18, in a schematic side view.

In order to perform, for instance, a cardiac catheterization in the patient 1 depicted in FIG. 1, an access into the Arteria femoralis communis 3, through which a catheter is advanced to the heart 300, is made in a thigh of the patient 1 at a site of puncture 2.

The puncture lies, as the exposed site of puncture in FIG. 2 shows, somewhat ventrally in the Arteria femoralis communis 3, which runs near the Vena femoralis 4 and is located between the inguinal ligament (ligamentum inguinale) 5 and the Arteria femoralis superficialis 6 and the Arteria profunda femoris 7.

In the creation of the access to the Arteria femoralis communis 3 and the introduction of the catheter into this artery, basically, the steps depicted schematically in the sectional images of FIGS. 3 through 7 are performed:

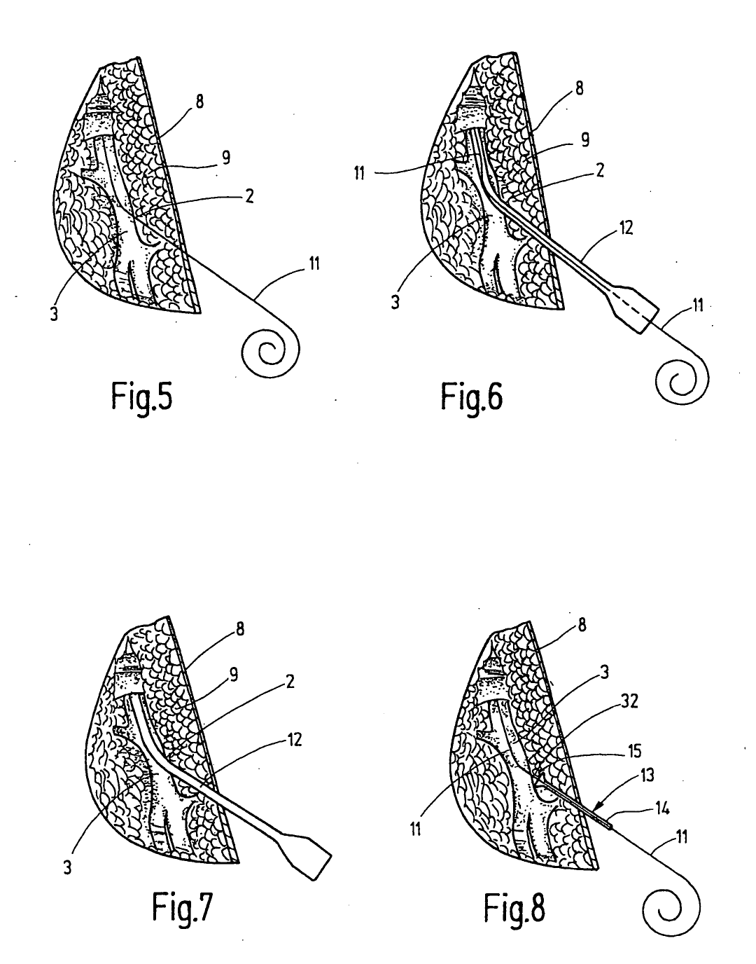

Through the skin 8 of the thigh and the underlying subcutaneous tissue 9, the artery wall is punctured at the site of puncture 2 with a hollow needle 10 (FIG. 3). Then, through the hollow needle (aspiration cannula) 10, a guide means in the form of a guide wire or a so-called Seldinger wire 11 is introduced into the artery 3 and advanced in the direction of the heart (FIG. 4). After the removal of the hollow needle 10 (FIG. 5), guided by the guide wire 11, a vascular sheath 12 is set in the artery 3 (FIG. 6), whereupon the guide wire 11 is pulled out (FIG. 7), such that the vascular sheath 12 is free for the introduction (not shown) of a diagnostic or therapy catheter. It should be noted here that basically the same procedure is also used for other peripheral vascular interventions. The catheter can also be placed brachially, for example. Cardiac catheterization is merely one illustrative example.

After finishing the catheterization, the catheter is removed, whereupon the vascular sheath 12 is removed. After removal of the vascular sheath 12, the puncture at the site of puncture 2 in the artery wall must be sealed, in order to

prevent bleeding with the complications mentioned in the introduction. In practice, this is usually accomplished in that, as already explained, after removal of the vascular sheath 12, the site of puncture is compressed by the physician or another trained individual by pressure on the skin of the thigh for a period of approximately 5 minutes or longer, until natural blood coagulation seals the puncture at the site of puncture. This is laborious and time-consuming for the physician or the trained individual and, moreover, for example, with adipose patience only inadequately possible.

This is where the invention starts, which provides a system for intracorporeal pressure maximization or for producing optimal external pressure for vascular occlusion at the site of puncture after artery catheterization. The new system works with an oblong occlusive means 13 that is placed, as will be explained, in the vicinity of the puncture at the puncture site 2 in the perivascular tissue. Pressure that compresses the perivascular tissue in the vicinity of the site of puncture and thus results in hemostasis is exerted on the occlusive means 13 from the outside. Through subsequent fixing of the occlusion means 13 at the skin level, the compression of the perivascular tissue is maintained for the required time without this requiring an additional or long-lasting action of the physician or of the trained individual.

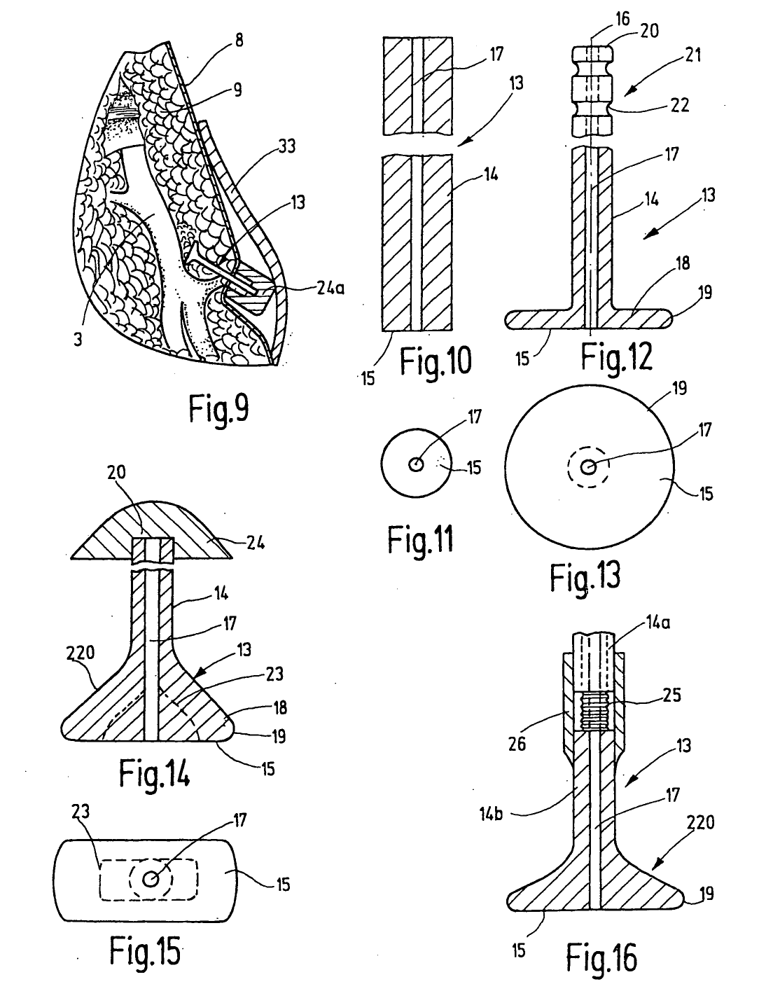

The occlusive means 13 is depicted in various embodiments in FIG. 9 through 19. Basically, it has an oblong shank 14, which is usually cylindrical and smooth walled and which bears on one end face a compression surface 15 which is oriented perpendicular to the shank's longitudinal axis 16. However,

embodiments are also conceivable in which the compression surface 15 is inclined at an angle deviating from 90° relative to the shank's longitudinal axis in order to obtain a better adaptation to the anatomical conditions of its use. The shank 14 has a continuous bore 17 that runs concentric to the shank's longitudinal axis 16 and opens in the area of the compression surface 15. In the embodiment according to FIG. 10, 11, the circular shaped compression surface 15 has the same diameter as the cylindrical shank 14. In contrast, in the embodiment according to FIG. 12, 13, the compression surface 15 is designed on the bottom of a flange-like region 18 with an enlarged cross-sectional surface formed on the shank 14. The flange-like region 18 is substantially disk shaped and is connected at an angle of 90° to the shank's longitudinal axis 17 to the shank 14, which thus substantially assumes the shape of a piston. The region 18 is rounded on its edge at reference number 19. The cylindrical shank 14 is provided in this case in a section 21 connected on the distal end 20 relative to the compression surface 15 with circumferential ribs 22 that are positioned at axial intervals and form predetermined breaking points. The shank 14 can, consequently, be varied in length in a simple manner by breaking at one of these predetermined breaking points.

The embodiment according to FIG. 14,15 is, in principle, similar to that according FIG. 12,13 but with the difference that the region 18 with an enlarged cross-sectional surface that carries the compression surface 15, transitions at reference number 220 over the length of the shank 14 continuously to the connected smooth-walled shank portion. Whereas in the embodiments explained according to FIG. 10 through 13, the compression surface 15 is circular, in the embodiment according to FIG. 14,15 it is designed oblong, as may be discerned, in particular from FIG. 15. Through this design of the outline of the compression surface 15, the area in which the perivascular tissue is compressed during the use of the occlusive means 13 is, if necessary, better adapted to the punctured artery. It should also be noted that with all embodiments of the occlusive means 13, the compression surface 15 can be designed circular, oblong, or with a different outline that proves advantageous for the respective use.

The compression surface 15 may have, in the region of the opening of the bore 17, a recess at least partially surrounding the opening of the bore, as is depicted with broken lines at reference number 23 in FIG. 14,15. This yields a ring-shaped compression surface 15 that results in corresponding ring-shaped pressure distribution in the compression of the perivascular tissue.

A cap 24 rounded on the top, which forms, for one thing, a sealing means for the bore 17 in the shank 14 and represents, for another thing, a widening on the distal shank end 20 that facilitates the fixing of the occlusion means 13 on the patient, as will be explained in detail, is placed on the shank 14. The cap 24 may even be connected unremovably to the shank 14, by being formed thereon, for example, whereby the then continuous bore 17 can be sealed by its own plug. The shape of the cap 24 is determined by the respective needs and anatomical conditions at the site of puncture. The shank widening formed thereby may, for example, also have a more plug-shaped cylindrical design, as depicted in FIG. 9 at 24a.

The embodiment according to FIG. 16 is substantially similar to that according to FIG. 14 but with the difference that the shank 14 has two coaxial shank sections 14a, 14b that are connected to each other by a threaded connection 25 that is covered toward the outside with a protective sleeve 26 that slides on or is connected to one of the shank parts 14a, 14b. The design enables adjustment of the shank length by rotating the shank part 14a selectively based on the needs of the respective application. In principle, it is also conceivable to omit the threaded connection 25 and design the two parts 14a, 14b to slide in each other telescopically in order to enable the desired variation in length of the shank 14.

Finally, the embodiment according to FIG. 17,18 likewise corresponds substantially to that according to FIG. 14, but here, in the region of the in this case circular compression surface 15 a torus-shaped, inflatable balloon 27 that can be inflated via a line 28 running through the bore 17 and by introduction of an inflation medium is provided. In the uninflated state, the balloon 27 is folded at least partially in the recess 23 provided in the region 18 with the enlarged diameter, whereby, optionally, it may even be partially accommodated in the bore 17. The balloon 17 [sic] makes it possible to increase the compression pressure exerted on the tissue with the occlusion means 13 already placed in the perivascular tissue and/or to control it precisely since the active compression surface 15a now lies on the bottom side of the balloon. Moreover, it is thus possible to enlarge the compression surface laterally.

During placement of the occlusion means in the tissue, the balloon 27 is deflated such that it does not interfere with the placement of the occlusion means.

Finally, FIG. 19 shows a sealing means designed as a plug 29 for the bore 17 in the shank 14. The plug 29 has a knob-like handle 30 and a cylindrical plug part 31 connected thereto that can be pressed sealingly into the bore 17 of the shank 14. The plug part 31 is usually just long enough that a secure hold in the bore 17 is ensured. However, embodiments are also conceivable in which, as shown in FIG. 19, it extends over the entire length or a substantial part of the length of the bore 17, in order to fill it completely and thus to prevent the development of a thrombus in the bore 17.

The function of the system according to the invention is clear from the following description of the handling of the occlusive means 13:

Referring to FIG. 7, after completion of the intervention, the intervention catheter is removed from the vascular sheath 12.

Starting from the state according to FIG. 7, the guide means in the form of an introduction wire or a Seldinger layer 11 is advanced again into the artery 3 via the vascular sheath 12, whereby the situation according to FIG. 6 is restored.

Now, the vascular sheath 12 is removed and the occlusive means 13 is advanced through the existing access channel to the site of puncture 2 in the vicinity of the site of puncture. At this time, the perivascular tissue 9 in the region of the site

of puncture 12 is locally compressed by the compression surface 15, as shown in FIG. 8 at 32. Now, the situation according to FIG. 8 is obtained, in which the shank 14 protrudes beyond the patient's skin 8 at the incision site. If necessary, a dilator may be used to facilitate the placement of the occlusive means 13.

The introduction wire or Seldinger wire 11 is now removed and the external pressure on the site of puncture 2 is maximized with the occlusive means 13 to minimize the escape of blood. At this time or already in a preparatory step, the length of the shank 14 of the occlusive means 13 is adapted to the respective anatomical conditions, i.e., substantially, the thickness of the perivascular tissue and of the subcutaneous tissue 9 in the region of the site of puncture 2, if an occlusive means 13 with an appropriate fixed shank length was not used from the outset.

Moreover, if necessary, the bore 17 on the distal shank end 20 is sealed either by means of the plug 29 (FIG. 19) or a cap 24,24a; and the occlusive means 13 is fixed at skin level by means of a bandage shown in FIG. 1,9 at 33 or a corresponding dressing. The widening on the shank end formed by the cap 24 or the knob 30 (FIG. 19) enables a large-area support of the occlusive means 13 on the dressing 33, whereby it simultaneously forms a support on the surface of the skin. The occlusive means 13 is thus further stabilized with regard to its position.

With the use of the occlusive means 13 according to FIG. 17, 18, after placement of the occlusive means 13, the balloon 27 is inflated, which, as already mentioned, enables enlarging the compression surface and increasing and/or delicately controlling the compression pressure without having to change the position of the occlusive means 13 itself.

The balloon 27 may also be introduced as a separate part like a balloon catheter through the bore 17 in the shank 14 and placed in front of the compression surface 15.

As already mentioned in the introduction, the system according to the invention is suitable for all vascular interventions in which a peripheral vascular access is made. The cardiac catheterization is merely used to illustrate the basic mode of action of the new system, as already has been mentioned.

The occlusive means 13 is, as a rule, manufactured from a biocompatible and medically approved plastic. Its dimensions are determined according to the requirements and the anatomical conditions of the respective application. As a nonrestrictive example, it is indicated here that the diameter of the bore 17 is preferably between 0.9 and 1.5 mm, depending on the diameter of the guide wire 11, and the diameter of the compression surface 15 is preferably within a range from (4) to 6 (8) to 9 mm. These dimensions apply with the use of a vascular sheath with the French size 5 to 11 (1.65 mm diameter to 3.7 mm diameter). The length of the shank 14 is preferably between 3 and 7 cm, but basically depends, as already mentioned, on the anatomical relationships of the patient in the area of the site of puncture.

1. System for occluding a blood vessel at a site of puncture of a blood vessel, especially after artery catheterization, comprises an oblong occlusive means (13) that has a shank (14) and a compression surface (15) configured thereon as the end face, whereby the shank comprises a continuous bore (17) that extends through the compression surface to accommodate at least one guide means (11) inserted into the blood vessel and is, on the distal end (20) opposite the compression surface, adapted to be fixed on the patient on the skin when the compression surface is advanced towards the site of puncture (2).

2. System according to claim 1, characterized in that the shank (14) of the occlusive means is designed with a substantially cylindrical shape.

3. System according to claim 1, characterized in that the shank (14) has a region (18) with an enlarged cross-sectional surface that carries the compression surface (15).

4. System according to claim 3, characterized in that the shank (14) is designed with a piston-like shape and has a substantially smooth-walled shank portion that connects to the region (18) with the enlarged diameter.

5. System according to claim 4, characterized in that the region (18) with the enlarged diameter, starting from the compression surface (15) configured on its end face transitions continuously over the length (220) of the shank into the smooth-walled shank portion.

6. System according to claim 1, characterized in that the compression surface (15) is substantially circular in shape.

7. System according to claim 1, characterized in that the compression surface (15) is oblong.

8. System according to one of the preceding claims claim 1, characterized in that the compression surface (15) runs substantially at a right angle to the longitudinal axis (16) of the shank (14).

9. System according to claim 1, characterized in that the compression surface (15) runs oblique to the longitudinal axis (16) of the shank at an angle deviating from 90°.

10. System according to claim 1, characterized in that the compression surface (15) has a recess (23) at least partially surrounding the opening of the bore (17).

11. System according to claim 1, characterized in that the shank (14) is designed variable in its length.

12. System according to claim 11, characterized in that the shank (14) is designed telescopically.

13. System according to claim 11, characterized in that the shank (14) has at least two parts (14a, 14b) connected to each other by screw threads (25).

14. System according to claim 11, characterized in that the shank (14) has predetermined breaking points (22) positioned at intervals relative to each other in the longitudinal direction of the shank.

15. System according to claim 1, characterized in that the shank has a widening on its distal end (20).

16. System according to claim 15, characterized in that the widening is designed with a cap-like shape (24).

17. System according to claim 15 or 16, characterized in that the widening is releasably connected to the shank.

18. System according to claim 1, characterized in that it has a sealing means (29) for the bore (17) of the shank (14).

19. System according to claim 18, characterized in that the sealing means is (24,29) adapted for attachment on the distal end of the shank (20).

20. System according to claim 18, characterized in that the sealing means has a part (31) insertable into the bore (17) of the shank (14), that substantially fills the bore over at least a section of its longitudinal extension.

21. System according to claim 1, characterized in that the occlusive means (13) has a balloon (27) in the region of the compression surface that is inflatable via a line (28) running through the shank.

22. System according to claim 21, characterized in that the balloon (27), in the uninflated state, is arranged at least partially in a recess (23) in the region of the compression surface (15) and/or in the bore (17) of the shank (14).

23. System according to claim 21, characterized in that the balloon (27) is designed to be introducible into the occlusive means through the bore (17) of the shank (14).
